(19) United States
(12) Patent Application Publication
(10) Pub. No.: US 2013/0109712 A1
(43) Pub. Date: May 2, 2013

(54) Statins for the Prevention or Treatment of Drug Addictions

(71) Applicants: Marcello Solinas, Poitiers (FR); Claudia Chauvet, Poitiers (FR); Mohamed Jaber, Buxerolles (FR)

(72) Inventors: Marcello Solinas, Poitiers (FR); Claudia Chauvet, Poitiers (FR); Mohamed Jaber, Buxerolles (FR)

(73) Assignees: Universite de Poitiers, Poitiers (FR); Centre National De La Recherche Scientifique, Paris (FR)

(21) Appl. No.: 13/641,195

(22) PCT Filed: Apr. 5, 2011
(86) PCT No.: PCT/IB2011/051457

§ 371 Date: Jan. 16, 2013

(30) Foreign Application Priority Data
Apr. 14, 2010 (FR) 10 01581

(51) Int. Cl. A61K 45/06; A61K 31/366

(52) U.S. Cl. CPC A61K 45/06; A61K 31/366
USPC 514/282; 514/460; 514/454; 514/326; 514/397; 514/279

(57) ABSTRACT

The present invention relates to a statin for the use thereof for the prevention or treatment of drug addictions or in stopping drug consumption.

### FIELD OF THE INVENTION

The present invention relates to medicaments for the use thereof in the prevention or treatment of drug addictions or in withdrawal from drug consumption.

### TECHNICAL BACKGROUND

The increase in the consumption of addictive drugs, such as cocaine, in the general population, and in particular in young adults, makes it necessary to develop strategies which facilitate withdrawal and which limit relapse in the event of withdrawal, in particular by reducing the desire for the drug, more commonly known as craving.

At the current time, two major types of treatments for addictions are proposed, pharmacological and nonpharmacological.

The nonpharmacological treatments essentially involve psychotherapy, in order to identify the possible imbalances responsible for the drug taking and to correct them, and also to modify the behavior of individuals with respect to the drug of addiction. However, the addiction is sometimes such that it is necessary to have recourse to additional means.

The inventors have thus proposed, on the basis of experiments carried out in mice, that environmental conditions that are enriched, in particular by social, physical and intellectual stimulations, could facilitate withdrawal in cocaine-addictive individuals (Solinas et al. (2008) Proc. Natl. Acad. Sci. USA 105:17145-17150). However, this treatment also has its limits, since recent studies by the inventors, confirmed by the studies of another team, indicate that, if an individual who has undergone withdrawal and been treated with an enriched environment takes cocaine, this could reinstate cocaine addiction (Chauvet et al. (2009) Neuropsychopharmacology 34:2767-2778; Thiel et al. (2009) Int. J. Neuropsychopharmacol. 12:1151-1156).

It has also recently been proposed, on the basis of experiments carried out in rats, that high-frequency electrical stimulation of the subthalamic nucleus, using intracerebral electrodes, could make it possible to reduce the craving for cocaine (Rouaud et al. (2010) Proc. Natl. Acad. Sci. USA 107:1196-200). However, while the invasiveness of this technique is acceptable in the case of serious neurodegenerative diseases, such as Parkinson's disease, this appears to be less justified in simple cases of drug addiction, and it appears that this technique should be reserved for cases of addiction that cannot be treated otherwise.

As regards the pharmacological treatments, the available pharmacopeia is relatively limited, in particular with regard to the treatment of cocaine addiction. In fact, for the latter, there is currently no real pharmacological treatment for addiction. Indeed, the pharmacological treatments used aim essentially to treat certain side effects of cocaine withdrawal, such as depression. Five compounds, approved for other indications, have shown a beneficial effect during clinical trials, namely disulfiram, modafinil, propranolol, topiramate, vigabatrin and baclofen, but they are not yet officially approved for the treatment of cocaine withdrawal (O'Brien (2005) Am. J. Psychiatry 162:1423-1431).

It therefore remains urgent to find a treatment which is an alternative, or supplementary, to the current treatments for improving the treatment of drug addictions.

Statins are inhibitors of HMG-CoA reductase, an enzyme of the cholesterol synthesis pathway. They are mainly indicated for reducing cholesterolemia in individuals in whom there is a risk of a cardiovascular event.

### SUMMARY OF THE INVENTION

The present invention ensues from the unexpected demonstration, by the inventors, that at least one statin, simvastatin, administered chronically (1 mg/kg i.p.) during a withdrawal period of 20 days to rats previously made cocaine-addictive or nicotine-addictive, made it possible to significantly reduce the craving for the drug after the withdrawal period, compared with control rats having received a simple vehicle instead of the simvastatin.

Thus, the present invention relates to at least one statin for use in the prevention or treatment of addiction to a drug or in the withdrawal from drug consumption in an individual.

The present invention also relates to the use of at least one statin for the preparation of a medicament intended for the prevention or treatment of addiction to a drug or in withdrawal from drug consumption in an individual.

The present invention also relates to a method of prevention or treatment of addiction to a drug or of withdrawal from drug consumption in an individual, in which a prophylactically or therapeutically effective amount of at least one statin is administered to the individual.

In a preferred embodiment of the statin for use as defined above, of the use of the statin as defined above and of the method defined above, the statin is in combination with at least one additional compound intended for the prevention or treatment of addiction to a drug or for withdrawal from drug consumption.

The present invention also relates to a pharmaceutical composition comprising, as active substances:
at least one statin, and
at least one additional compound intended for the prevention or treatment of addiction to a drug or for withdrawal from drug consumption, and
optionally, a pharmaceutically acceptable vehicle, preferably for use in the prevention or treatment of addiction to a drug or in withdrawal from drug consumption in an individual.

The present invention also relates to products comprising:
at least one statin, and
at least one additional compound intended for the prevention or treatment of addiction to a drug or for withdrawal from drug consumption, as a combination product for the simultaneous, separate or sequential use in the prevention or treatment of an addiction to a drug or in withdrawal from drug consumption.

### DESCRIPTION OF THE FIGURES

FIGS. 1A and 1B

FIGS. 1A and 1B represent the average levels of self-administration of cocaine in rats which will subsequently receive a chronic treatment of a control solution (control group) or a chronic treatment with simvastatin (simvastatin group). FIG. 1A represents the number of nose pokes for the active and inactive holes and FIG. 1B represents the number of cocaine injections during the 20 self-administration sessions. It should be noted that the chronic treatments of control solution or of simvastatin begin the day after the final self-administration session and end the day before the drug-seeking behavior tests.

FIGS. 2A and 2B

FIGS. 2A and 2B represent the decrease in the cocaine-seeking behavior in the rats treated chronically with simvastatin. The drug-seeking behavior is measured by the number of nose pokes in the active holes carried out by the rats during the 3 h extinction test session. The active nose pokes are not reinforced by a cocaine injection. FIG. 2A represents the number of nose pokes in the active holes during each hour of the extinction test. FIG. 2B represents the total number of nose pokes in the active holes during the 3 hours of extinction. The symbol ** indicates a significant decrease in the nose pokes of the simvastatin group compared with the control group.

FIGS. 3A and 3B

FIGS. 3A and 3B represent the average levels of nicotine self-administration in rats that will subsequently receive a chronic treatment of a control solution (control group) or a chronic treatment of simvastatin (simvastatin group). FIG. 3A represents the number of nose pokes for the active and inactive holes and FIG. 3B represents the number of nicotine injections during the 24 self-administration sessions according to a fixed ratio 1 or 3 (FR1 or FR3) schedule of reinforcement. It should be noted that the chronic treatments of control solution or of simvastatin begin the day after the final self-administration session and end the day before the drug-seeking behavior tests.

FIG. 4

FIG. 4 represents the decrease in nicotine-seeking behavior in the rats treated chronically with simvastatin. The drug-seeking behavior is measured by the number of nose pokes in the active holes carried out by the rats during the 1 h extinction test session. The active nose pokes are not reinforced by a nicotine injection. The symbol ** indicates a significant decrease in the nose pokes of the simvastatin group compared with the control group.

FIGS. 5A and 5B

FIG. 5A represents the average levels of self-administration of food in rats which will subsequently receive a chronic treatment of a control solution (control group) or a chronic treatment of simvastatin (simvastatin group) during 1-hour or 4-hour sessions. FIG. 5B represents the number of nose pokes for the active and inactive holes during the 15 self-administration sessions. It should be noted that the chronic treatments of control solution or of simvastatin begin the day after the final self-administration session and end the day before the food-seeking behavior tests.

### DETAILED DESCRIPTION OF THE INVENTION

As it is intended herein, an “addiction” to a drug is defined, in particular, by a drug consumption which is no longer controlled by the individual and which persists despite its possible negative consequences, which may be present or foreseeable, on the individual, in particular in an individual who is aware or who is informed of the possible negative consequences. According to the invention, the statins make it possible to avoid or limit the establishment of an addiction and to reduce or eliminate an addiction that has already established. Thus, in particular, the statins enable the individual in a situation of addiction to a drug to control, and in particular to stop, the consumption of the drug.

As it is intended herein, the “withdrawal” from drug consumption in an individual is defined, in particular, by the stopping of the consumption of the drug in an individual, in particular in an individual in a situation of addiction with respect to the drug. According to the invention, the statins facilitate withdrawal and make it possible, in particular, to reduce the relapse i.e. renewed drug consumption again, risk or probability of an individual who has undergone withdrawal or who is abstinent, i.e. who has stopped consuming a drug, in particular a drug with respect to which he was in a situation of addiction.

In particular, according to the invention, the statins make it possible to prevent or treat the surge to consume a drug, in particular in an individual who has undergone withdrawal from a drug with respect to which he was in a situation of addiction or on which he was dependent. The surge, often described as intense, to consume a drug is also denoted as a craving.

Preferably, the individual according to the invention is a human being. The individual according to the invention exhibits in particular a drug addiction or a dependence, or is in a state of addiction, with respect to the drug according to the invention.

As it is intended herein, the drugs according to the invention encompass all the compounds liable of inducing a dependence or an addiction in an individual consuming them. The drugs according to the invention can in particular be referred to as “an addictive drug” or “a drug of abuse”. The drug consumption according to the invention can be carried out by routes of any type, for example by ingestion, inhalation, or subcutaneous or intravenous injection.

The drugs according to the invention can in particular be narcotics, medicaments, tobacco or alcohol.

Preferably, the drugs according to the invention are selected from the group consisting of cocaine, crack, cannabis, morphine, opioids, heroin, ecstasy, LSD, amphetamines, ketamine, tobacco and alcohol.

As will be clear to those skilled in the art, tobacco addiction according to the invention is generally a nicotine addiction and alcohol addiction according to the invention is generally an ethanol addiction.

As it is intended herein, the term “cannabis” groups together all or any of the psychoactive cannabinoids, including in particular tetrahydrocannabinol (THC). The term “opioid”, for its part, denotes all or any of the psychoactive opioids or of the psychoactive derivatives of morphine.

Particularly preferably, the drug according to the invention is nicotine, cocaine or crack, in particular nicotine or cocaine. The term “cocaine” groups together, or denotes without distinction, cocaine in itself and also its various salts, such as cocaine hydrochloride.

The statins, or HMG-CoA reductase inhibitors, form a drug class well known to those skilled in the art. The statins are in particular described by Smith et al. (2009) Drug class review: HMG-CoA reductase inhibitors (statins). Update 5 (http://www.ohsu.edu/drugeffectiveness/reports/final.cfm). Moreover, within the meaning of the invention, a “statin” denotes both a statin in itself and a pharmaceutically acceptable salt of a statin.

Preferably, the statin according to the invention is selected from the group consisting of simvastatin, atorvastatin, fluvastatin, lovastatin, pravastatin and rosuvastatin.

Particularly preferably, the statin according to the invention is simvastatin.

The additional compound intended for the prevention or treatment of addiction to a drug or for withdrawal from drug consumption according to the invention may be of any type; however, it is preferably selected from the group consisting of methadone, buprenorphine, naltrexone, acamprosate, disulfiram, topiramate, bupropion, rimonabant, disulfiram, modafinil, propranolol, baclofen and ondansetron. For the pharmaceutical composition according to the invention, it is preferred that the additional compound intended for the prevention or treatment of addiction to a drug or for withdrawal from drug consumption according to the invention is not rimonabant, and more preferably, that it is selected from the group consisting of methadone, buprenorphine, naltrexone, acamprosate, disulfiram, topiramate, bupropion, disulfiram, modafinil, propranolol, baclofen and ondansetron. Moreover, when the drug according to the invention is tobacco, the additional compound intended for the prevention or treatment of addiction to a drug or for withdrawal from drug consumption according to the invention can be nicotine administered other than by inhalation of smoke originating from the burning of tobacco, in particular transdermally, for example by means of a patch, nasally, for example by means of a vaporizer, or orally, for example by means of a chewing gum. These additional compounds are in particular described in O'Brien (2005) Am. J. Psychiatry 162:1423-1431.

In particular, when the drug is cocaine or crack, the additional compound is preferably disulfiram, topiramate, modafinil, propranolol or baclofen. When the drug is nicotine, the additional compound is preferably bupropion or rimonabant, more preferably bupropion.

As it is intended herein, the expression “in combination” or “combination product” means that the statin according to the invention and the additional compound according to the invention can be combined within one and the same pharmaceutical composition, and therefore can be administered together, or else administered separately, i.e. according to distinct routes of administration and/or distinct administration regimens, with the proviso that, when they are administered separately, the respective periods of activity of the statin and of the additional compound totally or partly overlap.

Thus, when the statin according to the invention and the additional compound according to the invention are administered separately, the additional compound will preferably be administered within 24 hours, more preferably within 2 hours, and even more preferably within the hour, following the administration of the statin, and its administration will optionally be continued over the following days. The statin will, vice versa, preferably be administered within 24 hours, more preferably within 2 hours, and even more preferably within the hour, following the administration of the additional compound, and its administration will optionally be continued over the following days. In another preferred embodiment of the invention, when the statin and the additional compound according to the invention are administered separately, they are administered essentially simultaneously.

### EXAMPLES

The inventors used protocols of self-administration in rats in order to test the curative effects of simvastatin in a preclinical model. These protocols make it possible to mimic dependence in humans as closely as possible. Indeed, in the self-administration experiments, the rats can self-inject the drug intravenously by pressing a lever or by poking their nose in a hole. They thus control their drug consumption themselves. This model is particularly representative of the situation in humans since it includes drug-seeking and -taking factors identical to those observed in humans.

In particular, because relapse constituted by taking cocaine or nicotine after a long period of abstinence is one of the most difficult obstacles for curing addiction, the inventors focused on this phase of the addiction cycle by using a model of reinstatement of drug-seeking behavior after a period of forced withdrawal. In this model, the animals first learn to self-administer the drug and are subsequently kept in a phase of abstinence for a given period of time (from one day to several months), in order to measure the persistence of the craving for the drug at the time of the relapse test, during which the drug is not available (Epstein et al. (2006) Psychopharmacology (Berl.) 189:1-16).

Thus, the logic of the experiment is the following:
1) the rats acquire self-administration under similar conditions;
2) during withdrawal from self-administration, they are separated and receive, once a day for 20 days, an injection of simvastatin (1 mg/kg i.p) or a control solution;
3) they are tested in a drug-seeking protocol in which their responses are not reinforced by the drug.

Moreover, the inventors studied the effects of simvastatin on food-seeking behavior in order to evaluate the specificity of action of this compound.

### Materials and Method

Individuals

Adult male (11-12 weeks old) Sprague-Dawley rats (Janvier, France), experimentally naive at the beginning of the study, are placed in the animal house with controlled temperature and degree of humidity, and are kept in a 12-hour light/dark cycle (light active at 7 am). As soon as they arrive, the rats are placed in cages in groups of 3 for one week, before beginning the surgery, consisting of the implantation of a catheter in the jugular vein. After the surgery and during the rest of the experiment, the rats are placed in cages individually. All the experiments are carried out during the “day” phase, in accordance with EC regulations for the use of animals in research (86/609/EEC).

Catheterization

For the cocaine self-administration procedures, the catheters (Solinas et al. (2003) J. Pharmacol. Exp. Ther. 306:93-102) are implanted in the right jugular vein, under sterile conditions and under general anesthesia (intraperitoneal (i.p) injection of ketamine (60 mg/kg) and of xylazine (10 mg/kg)). A 20 mm plastic lock is implanted subcutaneously in the back of the rat. During the self-administration sessions, the catheter is connected to the injection pump via a metal tube which attaches to the plastic lock located in the back of the rat. The catheters are checked before and after each session with 0.1 ml of saline solution.

Cocaine self-administration apparatus and procedures

The self-administration equipment and procedures are similar to those described by Chauvet et al. (2009) Neuropsychopharmacology 34:2767-78.

The self-administration experiments are carried out in Imetronic cages equipped with nose-poke holes and controlled by Imetronic interfaces and software (Imetronic, Pessac, France; www.imetronic.com).

For the cocaine, 20 self-administration sessions are carried out using a fixed ratio 1 (or FR1) schedule of reinforcement, i.e., during the sessions, a single nose poke in the active hole immediately delivers an intravenous (i.v.) injection of cocaine (0.6 mg/injection). During the injection, the light is activated for 5 seconds, followed by an extinction period of 5 seconds during which the self-administration chamber is dark and the nose pokes have no consequence. A nose poke in the inactive hole has no consequence. The nose pokes in the active and inactive hole are recorded. All the self-administration sessions last 3 hours.

For the nicotine, 24 self-administration sessions are carried out using a fixed ratio 1 or 3 (FR1 or FR3) schedule of reinforcement, i.e., during the sessions, respectively one or three nose pokes in the active hole deliver an i.v. injection of nicotine. The nicotine doses delivered are 22.5 μg/injection or 7.5 μg/injection. During the injection, the light is activated for 5 seconds, followed by a period of extinction for 5 seconds during which the self-administration chamber is dark and the nose pokes have no consequence. A nose poke in the inactive hole has no consequence. The nose pokes in the active and inactive hole are recorded. All the self-administration sessions last 1 or 3 hours. During sessions 1-5, the rats can self-administer a nicotine dose of 22.5 μg/injection according to the FR1 protocol; during sessions 6-7, the rats can self-administer a nicotine dose of 22.5 μg/injection according to the FR3 protocol; during sessions 9-14, the rats can self-administer a nicotine dose of 7.5 μg/injection according to the FR1 protocol; and during sessions 15-24, the rats can self-administer a nicotine dose of 7.5 μg/injection according to the FR3 protocol.

For the food, 15 self-administration sessions are carried out using an FR1 schedule of reinforcement in which a single nose poke in the active orifice immediately delivers a 45 mg granule of food. During the food delivery, the light is activated for 5 seconds, followed by a period of extinction of 5 seconds during which the self-administration chamber is dark and the nose pokes have no consequence. A nose poke in the inactive hole has no consequence. The nose pokes in the active and inactive hole are recorded. The self-administration sessions last 1 or 4 hours.

Long period of abstinence and chronic treatment with simvastatin

At the end of the final self-administration session, the rats are divided up, in a pseudorandomized manner, into 2 groups, each having similar levels of drug or food self-administration: one group receives simvastatin at the dose of 1 mg/kg i.p. and the other group receives equivalent volumes of control solution. The rats are kept abstinent for 20 days, during which they receive the daily injection of simvastatin or of control solution (the injections are given between 2 pm and 4 pm each day).

This period of abstinence allows the development of a phenomenon known as incubation of craving, which is reflected by an increase in the craving to consume drugs over time (Grimm et al. (2001) Nature 412:141-2).

Importantly, the final simvastatin injection is administered the day before the drug-seeking behavior test, in order to avoid potential direct effects of the drug.

Cocaine-seeking test

After 20 days of treatment with simvastatin, the rats are placed in the self-administration room and tested for the drug-seeking or food-seeking behavior in an extinction session of 3 hours or of 1 hour in the case of nicotine. The nose pokes in the active holes during this extinction phase produce the same stimuli (light and noise of the pump) that were present during the self-administration phase, but the syringes are removed from the injection pumps and the cocaine and the nicotine are not delivered. For the food, the granules are removed from the distributor and are not therefore supplied. The 3-hour extinction session is divided up into 3×1 hour of extinction (or 1 hour only for nicotine). The number of nose pokes in the active holes is used as a measure of the drug-seeking behavior.

Drugs

The cocaine is purchased from COOPER (Cooperation Pharmaceutique Francaise, France; www.cooper.fr), the nicotine is purchased from Sigma/Aldrich, and the simvastatin is a gift from the Ranbaxy laboratory, India. The cocaine and the nicotine are dissolved in a sterile saline solution (0.9%). The simvastatin solution is prepared daily in the following way: 5 mg of simvastatin are dissolved in 250 μL of ethanol and 250 μl of tween 80, then a sterile saline solution is added up to a final volume of 15 ml. Thus, a solution containing 0.33 mg/ml of simvastatin is obtained. It is injected at a volume of 3 ml/kg for a final dose of 1 mg/kg. The control solution is identical but does not include simvastatin.

### Example 1

### The Chronic Treatment with Simvastatin Reduces the Cocaine Seeking

Self-administration of cocaine before treatment with simvastatin or with the control

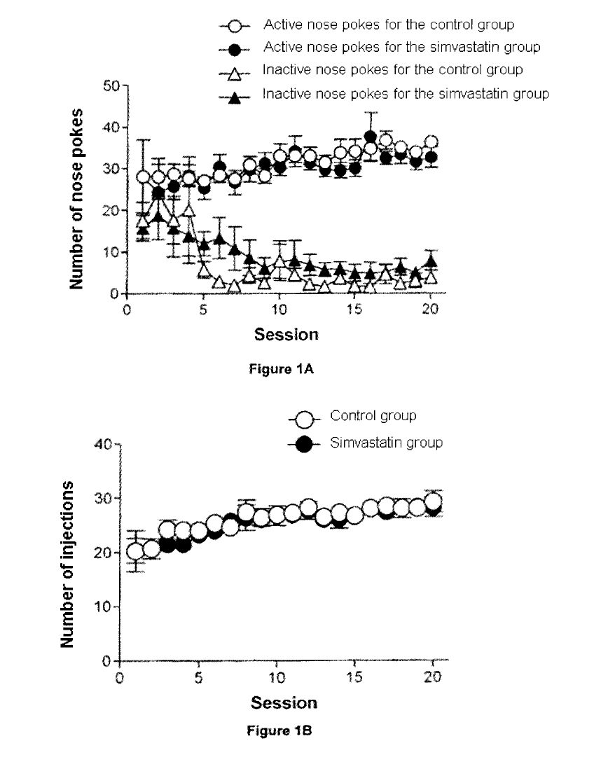

At the end of the self-administration period, each of the 2 groups which is to receive, respectively, simvastatin and the control exhibits similar levels of cocaine self-administration. Thus, the number of active and inactive nose pokes does not differ between the future simvastatin and control groups (FIGS. 1A and 1B).

The chronic treatment with simvastatin reduces the cocaine seeking

The rats having self-administered cocaine and having subsequently received the control solution during the 20-day abstinence period exhibit a high level of drug-seeking behavior during the test (approximately 350 active nose pokes). In comparison, rats which never self-administered cocaine do not produce more than 20 active nose pokes during the same test.

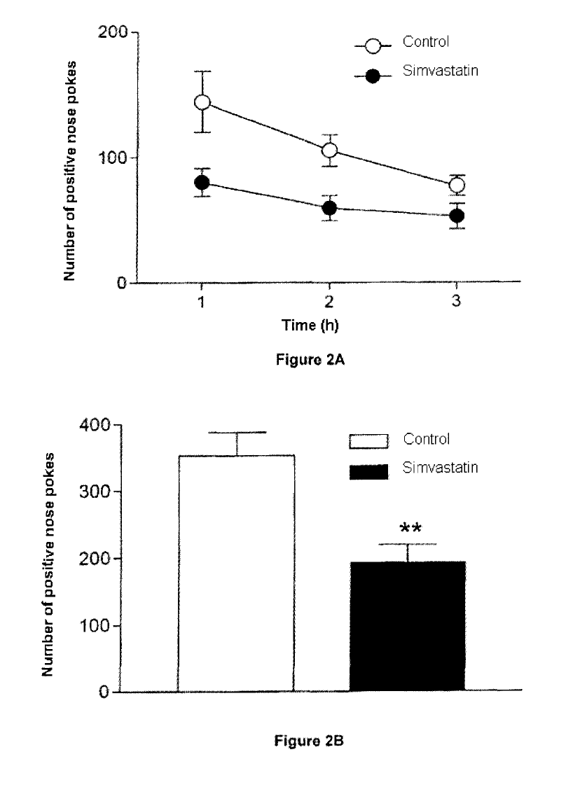

The rats having self-administered cocaine and having subsequently received daily injections of 1 mg/kg of simvastatin during 20 days of abstinence show a significant reduction in drug-seeking behavior (approximately 190 active nose pokes, i.e. a 45% reduction) (FIGS. 2A and 2B).

### Example 2

### The Chronic Treatment with Simvastatin Reduces the Nicotine Seeking

Self-administration of nicotine before the treatment with simvastatin or with the control

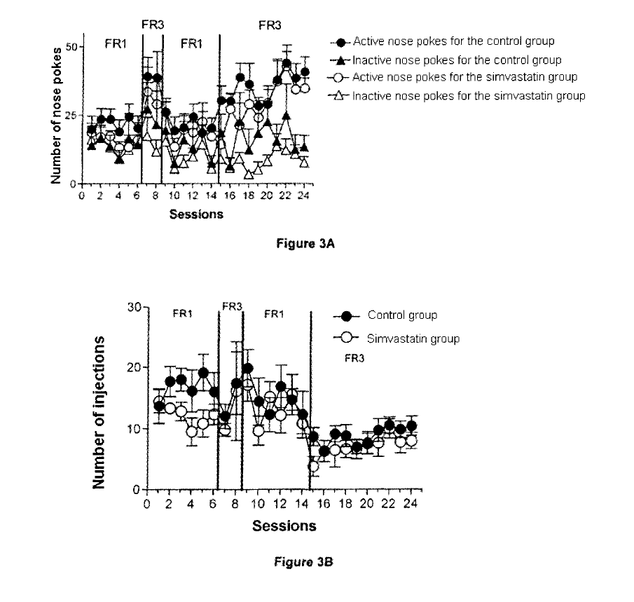

At the end of the self-administration period, each of the two groups which are to receive, respectively, simvastatin and the control, exhibit similar levels of nicotine self-administration. Thus, the number of active and inactive nose pokes does not differ between the future simvastatin and control groups (FIGS. 3A and 3B).

The chronic treatment with simvastatin reduces the nicotine seeking

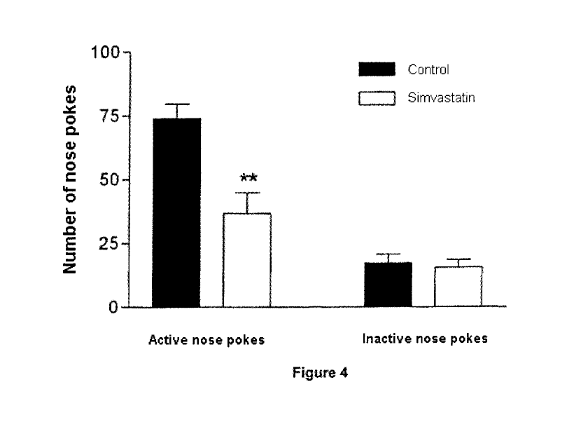

The rats having self-administered nicotine and having subsequently received the control solution during the 20-day abstinence period exhibit a high level of drug-seeking behavior during the test (approximately 100 active nose pokes). The rats having self-administered nicotine and having subsequently received daily injections of 1 mg/kg of simvastatin during 20 days of abstinence show a significant reduction in drug-seeking behavior (approximately 50%) (FIG. 4).

### Example 3

### The Chronic Treatment with Simvastatin does not Affect Food Seeking

Self-administration of food before the treatment with simvastatin or with the control

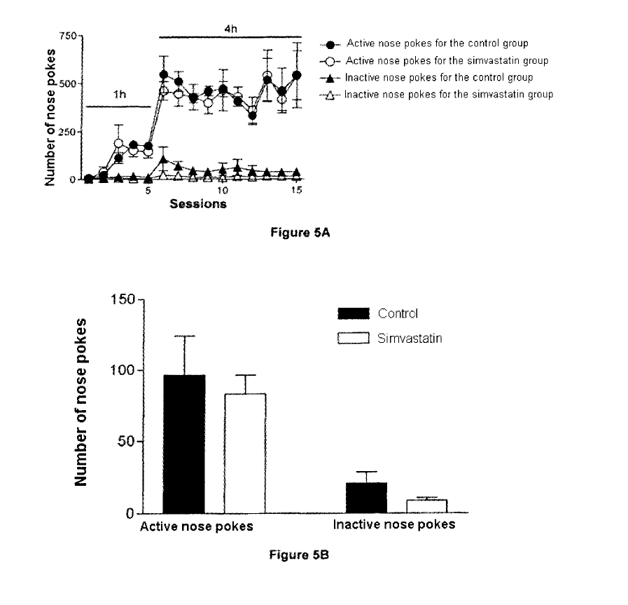

At the end of the self-administration period, each of the two groups which are to receive, respectively, simvastatin and the control exhibit similar levels of food self-administration. Thus, the number of active and inactive nose pokes does not differ between the future simvastatin and control groups (FIG. 5A).

The chronic treatment with simvastatin does not modify food seeking

There is no significant difference in the food-seeking behavior of the control rats and of the rats having received simvastatin (FIG. 5B).

Consequently, the chronic treatment with simvastatin does not modify food-seeking behavior, which suggests that simvastatin acts specifically on the effects of drugs of addiction and not on those of natural rewards.

1. A method for preventing or treating addiction to a drug or withdrawal from drug consumption comprising administering to an individual in need thereof a statin.

2. The method according to claim 1, wherein the statin is simvastatin, atorvastatin, fluvastatin, lovastatin, pravastatin, rosuvastatin, or combinations thereof.

3. The method according to claim 1, wherein the statin is simvastatin.

4. The method according to any of claim 1, wherein the drug cocaine, crack, cannabis, morphine, opioids, heroin, ecstasy, LSD, amphetamines, ketamine, tobacco, alcohol, or combinations thereof.

5. The method according to any of claim 1, wherein the drug is cocaine or nicotine.

6. The method according to any of claim 1, wherein said method is to prevent or treat in a craving to consume a drug.

7. The method according to any of claim 1, further comprising at least one additional compound capable of preventing or treating an addiction to a drug or withdrawal from drug consumption.

8. The method according to claim 7, wherein the additional compound is methadone, buprenorphine, naltrexone, acamprosate, disulfiram, topiramate, bupropion, rimonabant, disulfiram, modafinil, propranolol, baclofen, ondansetron, or combinations thereof.

9. A pharmaceutical composition comprising, as active substances:
at least one statin,
at least one additional compound capable of preventing or treating addiction to a drug or withdrawal from drug consumption, with the exception of rimonabant.

10. The pharmaceutical composition according to claim 9, wherein the additional compound is methadone, buprenorphine, naltrexone, acamprosate, disulfiram, topiramate, bupropion, disulfiram, modafinil, propranolol, baclofen, ondansetron, or combinations thereof.

11. A pharmaceutical composition comprising, as active substances:
at least one statin,
at least one additional compound capable of preventing or treating addiction to a drug or withdrawal from drug consumption.

12. The pharmaceutical composition according to claim 11, wherein the additional compound is methadone, buprenorphine, naltrexone, acamprosate, disulfiram, topiramate, bupropion, rimonabant, disulfiram, modafinil, propranolol, baclofen, ondansetron, or combinations thereof.

13. The pharmaceutical composition according to claim 9, wherein the composition is in a form capable of being administered simultaneously, separately or sequentially.

14. (canceled)

15. A method for preventing or treating addiction to a drug or withdrawal from drug consumption comprising administering to an individual in need thereof the pharmaceutical composition of claim 9.

16. The method according to claim 4, wherein the tobacco is nicotine.

17. The method according to claim 4, wherein the alcohol is ethanol.

18. The pharmaceutical composition according to claim 9, wherein the statin is simvastatin, atorvastatin, fluvastatin, lovastatin, pravastatin, rosuvastatin or combinations thereof.

19. The pharmaceutical composition according to claim 19, wherein the statin is simvastin.

20. The pharmaceutical composition according to claim 11, wherein the composition is in a form capable of being administered simultaneously, separately or sequentially.

21. The pharmaceutical composition according to claim 9, further comprising a pharmaceutically acceptable vehicle.

22. The pharmaceutical composition according to claim 11, further comprising a pharmaceutically acceptable vehicle.
